(19) United States
(12) Patent Application Publication
(10) Pub. No.: US 2013/0011825 A1
(43) Pub. Date: Jan. 10, 2013

(54) METHODS AND SYSTEMS FOR REDUCING DNA FRAGMENTATION IN A PROCESSED SPERM SAMPLE

(75) Inventors: Juan F. Moreno, College Station, TX (US); Kenneth Michael Evans, College Station, TX (US); Michael Kjelland, Valley City, ND (US); Jamie Gosalvez, Colmenar Viejo (ES); Clara Gonzalez-Martin, Navasota, TX (US); Carmen Lopez-Fernandez, Colmenar Viejo (ES)

(73) Assignee: INGURAN, LLC, Navasota, TX (US)

(21) Appl. No.: 13/635,833

(22) PCT Filed: Dec. 30, 2010
(86) PCT No.: PCT/US10/62598

§ 371 Date: Sep. 18, 2012

Related U.S. Application Data
(60) Provisional application No. 61/320,183, filed on Apr. 1, 2010.
(60) Provisional application No. 61/324,192, filed on Apr. 14, 2010.

(51) Int. Cl. C12N 5/076; G01N 21/64

(52) U.S. Cl. 435/2; 435/6.1

(57) ABSTRACT

A method and system for processing reproductive cell samples and for sorting sperm with reduced levels and occurrences of DNA fragmentation compared to conventional sorting and processing methods, and for using reproductive cell and sperm cell samples with low levels of DNA fragmentation to improve viability of insemination samples fertility and success rates of assisted reproductive procedures, including artificial insemination, in vitro fertilization, intracytoplasmic injection, and other related techniques.

This application is a national stage entry of, and claims priority to, International Patent Cooperation Treaty Application No. PCT/US10/062598, which is a continuation in part of, and claims priority to each of U.S. Provisional Patent Application 61/320,183, filed on Apr. 1, 2010, U.S. Provisional Patent Application 61/324,192, filed on Apr. 14, 2010 and International Patent Cooperation Treaty Application No. PCT/US10/54549, filed on Oct. 28, 2010, each are hereby incorporated herein by reference.

### FIELD

The present embodiments generally relate to methods and systems for reducing the number of DNA fragmentation events in various processed populations of cells and sperm cells, and more particularly, to modifying cell handling and sperm sorting processes to reduce DNA fragmentation events in various cell and sperm suspensions, for reducing the rate at which DNA fragmentation occurs in various cell and sperm samples, and for improving the overall success rates of assisted reproductive technologies and procedures.

### BACKGROUND

Sperm and sex sorted sperm (sperm sorted based on carrying an X or Y chromosome) are biological materials of great interest for assisted reproduction, particularly in the livestock breeding industry. However, damaged and/or dead sperm lack the viability for producing offspring through artificial insemination (AI), in vitro fertilization (IVF), Intracytoplasmic Sperm Injection (ICSI), embryo transfer (ET), or other assisted reproductive procedures. Damaged cells can include cells with altered membranes, cells undergoing apoptosis as well as cells with DNA fragmentation. A damaged sperm, when present in a viable sperm population used in an assisted reproductive procedure, may be capable of fertilizing an egg, but may fail to produce a viable embryo or may produce an embryo having genetic abnormalities that will not develop properly or may die later. In this way, sperm with DNA fragmentation could compete with viable sperm and reduce the overall likelihood of a successful pregnancy and increase the likelihood of producing malformed offspring. Simon et al., Human Reproduction, Vol. 25 No. 7 pp. 1594-1608 (2010), demonstrate the negative impact of increased rates of sperm DNA fragmentation on pregnancy rates and embryonic development following assisted reproductive procedures, such as in IVF and ICSI. Therefore, a need exists for methods and systems relating to sperm and other reproductive cell processing which reduce the levels of DNA fragmentation or the rate of DNA fragmentation in a processed reproductive cell or sorted sperm subpopulation and particularly in sorted subpopulations used in assisted reproductive procedures. More particularly, this need exists for processing conventional sperm and sex sorted sperm.

Sex sorted sperm are gender enriched subpopulations of sperm characterized and sorted on the basis of carrying either an X-chromosome or a Y-chromosome. The use of sex sorted sperm can particularly benefit the dairy and beef industries by providing offspring of the desired gender with a high degree of certainty. Most sperm sorting methods use flow cytometry and procedures that generally incorporate a non-toxic DNA binding fluorescent dye, which under the proper conditions, permeates the cell membrane and associates with the DNA of the cell in a stoichiometric manner. The amount of the dye associated with each sperm is closely related to the amount of DNA contained within each cell, which upon laser excitation, causes distinguishable fluorescence patterns in sperm bearing Y-chromosomes and those bearing X-chromosomes. U.S. Pat. Nos. 5,135,759, 6,357,307, 7,371,517 and 7,758,811, each of which are incorporated herein by reference, provide systems and methods adapted to sorting sperm based on the differences in the X- and Y-chromosomes.

While fresh ejaculates of all animals inherently contain a certain baseline number of sperm having DNA fragmentation, the overall level of DNA fragmentation in ejaculates from various individual donors can vary due to factors such as oxidative stress, apoptosis, failure in the histone-protamine replacement cycle and other environmental factors associated with semen production. Some of these DNA damaging factors can be compounded during subsequent sperm processing. In addition to this and given that sperm are generally delicate cells, sperm samples, after ejaculation that are handled ex vivo, can suffer additional iatrogenic damage throughout most sperm processes while preparing the sample for insemination. In particular, the methodology of sex sorting sperm includes several steps that produce stresses on the cells that are not only damaging, but may contribute to and intensify potential iatrogenic damage. Since the damage created in certain steps of the sorting process can be compounded by chemical and physical stresses endured throughout the sorting process, there is a particular need to minimize the occurrence of DNA fragmentation events in the sperm population as it is processed, sorted and stored. Therefore, a need exists for methods and systems to improve the viability of processed sperm samples by reducing the occurrence of DNA fragmentation in a population of sorted sperm, and in improving the success rate of artificial reproductive insemination technologies and the development of healthy offspring using sex sorted sperm.

### SUMMARY OF THE INVENTION

The present embodiments generally relate to cell sorting methods using flow cytometry and microfluidic devices for sorting sperm and other reproductive cells, and producing sperm populations exhibiting reduced amounts of DNA fragmentation or reduced rates of DNA fragmentation as compared to original cell or sorted sperm samples produced using conventional sorting methods. These sperm populations with reduced amounts of DNA fragmentation or reduced rates of DNA fragmentation are advantageous for improving successful birth rates using assisted insemination and/or fertilization techniques, such as AI, ICSI, IVF, ET and other related techniques.

Accordingly, embodiments disclosed herein provide methods and systems for reducing the overall level of DNA fragmentation in processed cell and sorted sperm samples.

In one aspect, embodiments disclosed herein provide a method and system for reducing DNA fragmentation in a cell or sperm population by the reduction of bacterial contamination in the original cell, semen or processed cell samples, also referred to as bacterial infection (BI) in this disclosure.

In another aspect, embodiments disclosed herein relate to methods and systems for reducing the rate of DNA fragmentation in processed cell samples, such as sperm samples or sex sorted sperm samples, by controlling the acidity of the sample in a stepwise manner.

In yet another aspect, embodiments disclosed herein provide a method and system for sex sorting sperm with modified processes which reduce levels of DNA fragmentation compared to previous sorting and handling methods.

In still another aspect, embodiments disclosed herein relate to the modification of a sperm staining process to reduce DNA fragmentation and preserve sperm viability by adding a quenching dye at an elevated pH.

In another aspect, embodiments disclosed herein relate to modifying staining procedures with a new quenching dye. Surprisingly, it has been found that yellow food dye, and more particularly yellow 6 provides a benefit in resolution when separating sperm and requires less Hoechst stain. By requiring less stain, the health of sperm is improved at the end of the sorting process.

### A BRIEF DESCRIPTION OF THE DRAWINGS

FIG. 1A illustrates a plotted representation of forward angle fluorescence versus side angle fluorescence of sperm in a flow cytometer.

FIG. 1B illustrates a side of containing sperm in a chromatin dispersion test, where the sperm were collected from a particular sort region.

FIG. 1C illustrates a side of containing sperm in a chromatin dispersion test, where the sperm were collected from a particular sort region.

FIG. 2A illustrates a plotted representation of forward angle fluorescence versus side angle fluorescence of sperm in a flow cytometer.

FIG. 2B illustrates a gated portion of sperm plotted as forward fluorescence versus integrated forward fluorescence in a flow cytometer.

FIG. 3A illustrates a graphical representation of DNA fragmentation over time in conventional sperm samples.

FIG. 3B illustrates a graphical representation of DNA fragmentation over time in sex sorted sperm samples.

FIG. 3C illustrates a graphical representation of the mean DNA fragmentation over time in conventional samples and sex sorted sperm samples.

FIG. 4 illustrates a flow cytometer in accordance with certain embodiments presented herein.

FIG. 5A illustrates a box and whisker plot illustrating the percentage of DNA fragmentation in sperm samples at different times in samples which did not exhibit bacterial infections.

FIG. 5B illustrates a graphical representation illustrating the percentage of DNA fragmentation in sperm samples at different times in samples which did not exhibit bacterial infections.

FIG. 5C illustrates a box and whisker plot illustrating the percentage of DNA fragmentation in sperm samples at different times in samples which exhibited bacterial infections.

FIG. 5D illustrates a graphical representation illustrating the percentage of DNA fragmentation in sperm samples at different times in samples which exhibited bacterial infections.

FIG. 6 illustrates a chart of DNA fragmentation over time for several different Red TALP treatments.

FIG. 7A and B illustrate forward fluorescence vs. side fluorescence plots from sample ejaculate.

FIG. 8A and B illustrate histograms of peak forward fluorescence in a flow cytometer generated from gating the R1 region of samples 7A and 7B respectively.

### DETAILED DESCRIPTION OF THE PREFERRED EMBODIMENTS

Before explaining the methods and systems in detail, it is to be understood that the methods are not limited to the particular embodiments described herein and can be carried out in a variety of ways. Furthermore, the methods and systems described are disclosed in a general fashion, so they may be applied to specific systems once the general principals are understood.

Some embodiments relate to a method for sorting cells that generate discrete subpopulations, where some subpopulations are enriched for particular characteristics and where the sorted subpopulations contain less cellular DNA fragmentation compared to sperm sorted by prior methods. Generally, a sperm sample, is directly or indirectly acquired from a mammal, including without limitation, those listed by Wilson, D. E. and Reeder, D. M., Mammal Species of the World, Smithsonian Institute Press (1993), the entire text of which is incorporated herein by reference. These mammals include, but are not limited to, bovine, equine, porcine, canine, feline, dolphin, goat, ovine, and corvine. In one embodiment, neat semen, which is freshly collected semen, can be processed by dilution or centrifugation with extenders or buffer solutions known in the art for preserving the motility and fertility of sperm in an extended sperm sample. In another embodiment, the sperm sample can be established by thawing previously cryopreserved and/or previously processed sperm from straws. In yet another embodiment, the sperm sample can comprise sperm heads removed from their respective sperm tails.

In one embodiment, an antibacterial agent, such as a quinolone, can be combined with a cell or sperm sample to minimize growth of bacteria and other microbes in the seminal plasma and on the surface of the sperm. Quinolones are a group of nalidixic acid and/or chloroquin derivatives including, but not limited to, ciprofloxacin, pipemidic acid, oxolinic acid and cinoxacin. Inhibition of bacterial growth in sperm samples has been correlated with a decrease in the level of DNA fragmentation in the sperm samples. In some embodiments, the quinolone can be ciprofloxacin from the fluoroquinolone group and is added as the primary antibacterial compound. In other embodiments, a quinolone cocktail comprising one or more antibiotics with at least one quinolone, can be added directly to a sperm or semen sample, to a buffer solution, to an extended sperm sample, to a staining media, or to another media used in processing sperm.

The sperm suspension containing the antibiotic or antimicrobial agent can be equilibrated and evenly dispersed by, incubation, mixing, or by other methods. A selected quinolone can be present in the target cell or sperm suspension at a range of about 0.05 μg/ml to about 20 μg/ml, about 0.2 μg/ml to about 5 μg/ml, and/or about 0.1 μg/ml to about 2 μg/ml. Two or more quinolones may each be added at the designated concentrations. Processed or sorted sperm can be collected directly into a media containing the quinolone, or quinlone cocktail. Alternatively, the quinolone or quinolone cocktail can be added subsequently, including: before sorting, after sorting, or even after sorting, freezing and thawing.

The buffer solution can include one or more buffer systems and may be selected from a non-exhaustive list including: TRIS, sodium citrate, egg yolk, milk, TALP, MOPS, HEPES based buffer, phosphate, borate, a bicarbonate, fluoride, a buffer containing BSA, and combinations thereof.

The method or system may further comprise a mechanism to adjust or optimize the combination of the quinolone, based on the specific composition of a given sperm sample, so that the level of quinolone is coordinated to maximally inhibit the growth of bacteria in the specific sperm sample. One example of such an adjustment or optimization may involve monitoring and adjusting the acidity of the specific sperm sample to reduce the level of DNA fragmentation in the sperm suspension.

The obtained sperm sample can then be previously cryopreserved and/or previously sex sorted. In this embodiment the quinolone, or quinolone cocktail, can be added in either a post thaw step or a post sort processing step. In one embodiment, cryopreserved sperm can be thawed and the quinolone can be added to the thawed sperm sample. This can be in conjunction with and before, or after, sorting, such as sex sorting. In another embodiment, the sperm sample can be cryopreserved after the introduction of quinolone. In each instance the processed sperm can be used to establish an insemination sample, whether conventional or sex sorted.

In another embodiment, the quinolone, or quinolone cocktail, can be applied to an IVF process or medium to reduce bacterial contamination in embryo suspensions.

In some embodiments, the quinolone is added to effectuate a reduction in the number of cells that exhibit DNA fragmentation which can reduce the overall level of DNA fragmentation in the cell suspension. The cell suspension may be any type of semen sample, such as fresh, frozen, conventional, sorted, or an oocyte or oocyte derivative suspension including, but not limited to oocytes, enucleated cells and injected derivatives thereof, intracytoplasmically injected oocytes, fertilized embryos and other related reproductive cell suspensions.

Certain aspects herein relate to an extended sperm sample which can include sperm, quinolone, and/or a buffer solution. In one embodiment, the quinolone can comprise a fluoroquinolone, such as ciprofloxacin, but other quinolones can also be used. The quinolone can be present in a range of about 0.05 μg/ml to about 20 μg/ml, about 0.2 μg/ml to about 5 μg/ml, and/or about 0.1 μg/ml to about 2 μg/ml in a fresh or extended sperm sample. The buffer solution can be any of those described above, as well as any combination thereof.

The sperm in the extended sperm sample can comprise sperm sorted for a fertility characteristic, such as the presence of an X-chromosome or Y-chromosome.

In another aspect, certain embodiments are related to a method of disinfecting or reducing the level of bacteria and other microbes in contaminated cell samples or cell culture media. This method can include the step of obtaining a cell sample including reproductive cells and applying one or more quinolones to the cell sample or the cell culture media to recover cell lines that have minimal or have no detectable levels of bacterial contamination. The reproductive cells can include sperm, oocytes, eggs, enucleated gametes with and without injected DNA, embryos, cultured embryos which can be treated by the addition of one or more quinolones and incubated to effectuate the anti-microbial effect of the quinolone.

In another aspect, certain embodiments relate to a method for producing an embryo which can include the steps of obtaining a sperm sample, combining the sperm sample with a quinolone, inhibiting bacterial growth in the sperm sample, and fertilizing an egg with sperm. The sperm can be sex sorted with a variety of staining and sorting methods, including but not limited to, those standard in the art as previously described.

In another aspect, the method can relate to a method for processing a sperm sample with a modified staining procedure. The method can begin with the step of obtaining sperm, which can be any of the sperm suspensions previously described, including previously frozen sperm samples. The sperm suspension can then be stained with a DNA selective dye in a first medium, and subsequently stained by addition of a second medium which may have a second dye. The pH of the second dye can be coordinated with the pH of the first dye, or the pH of the first dye in combination with the pH of the sperm sample to either maintain or adjust the pH to a desired range.

Coordinating the pH of the second dye should be understood to include for instance: matching the pH of the second medium to the pH of the first media; shifting the pH of the first medium with a suitable buffer system to achieve a staining effect, then readjusting the pH to a more suitable pH to minimize cell damage; or shifting the pH of the first medium with addition of the sperm sample to arrive at a desired final pH. The step of coordinating the pH of the second media can be accomplished by either reducing the potential pH changes and shocks to the sperm being stained, or by arriving at or near a final target pH. The second medium or buffer system may contain a second quenching dye to facilitate sorting.

The step of coordinating the second medium can comprise adjusting the pH of the second medium to be above 5.5, between about 5.5 and about 7.4, to between about 6.4 and about 7.4, about 6.4 and about 7.4. The pH of the second medium can be coordinated within 2 pH units of the first medium, within 1 pH unit of the first medium, or to have substantially the same pH as the first medium. Similarly, the second medium can be coordinated to arrive at a final sperm sample pH of between about 6.6 and about 7.2.

Previously, it was not appreciated that the application of a pH 5.5 medium including the second dye could be killing sperm within the suspension. As the pH 5.5 TALP contacts portions of the solution and begins mixing, but before the suspension reaches equilibrium, localized concentrations of the more acidic, lower pH mixture actually contacts a small localized subpopulation of sperm and damage or kill those localized sperm, reducing overall sperm motility and viability of the sperm sample. In this way, the process step of adding a pH 5.5 medium can produce unexpected negative effects on the sperm. Surprisingly, this effect can be even more damaging than applying the quenching dye at a higher pH. Conventionally, it was believed applying the quenching dye at a higher pH would result in a final suspension at a pH which was too high for the health of the sperm. For the purposes of this disclosure, one skilled in the art will recognize that the designation of pH, e.g. 5.5 pH, means the same as pH 5.5.

The first medium can contain a TALP based buffer, or another buffer, in combination with a DNA selective dye, such as the fluorescing DNA selective dye, Hoechst 33342, or other dyes such as those described herein.

The second medium can comprise a red TALP, which can comprises a TALP based buffer with red food dye. The second medium can include a quenching dye. The quenching dye can be a red food dye, yellow food dye, percoll, propidium iodide, trypan blue, or other dyes known to permeate compromised cell membranes for quenching fluorescing dyes.

The processed sperm can then be sex sorted, as previously described, and extended in a buffer selected from: TRIS, sodium citrate, egg yolk, milk, TALP, MOPS, HEPES, phosphate, KMT, borate, bicarbonate, a buffer containing BSA and/or fluoride, combinations thereof, or other known buffers for sperm processing or storage.

In one embodiment, the step of staining with both the first medium and the second medium can be accomplished in a single pH adjusting event. In another embodiment, the stained sperm can be sex sorted then cryopreserved.

In some embodiments, where it may be desirable to sex sort the sperm sample, the sperm sample can be stained with a marker, such as a fluorescent dye. The marker can be a DNA selective dye, such as Hoechst 33342, Hoechst 33258, BBC, SYBR-14, SYBR Green I, a bisbenzimide dye, or a combination thereof. In order for sperm to uptake a DNA selective dye, such as Hoechst 33342, uniformly and in a reasonable amount of time, the staining procedure requires the elevation of the sperm temperature and pH. The temperature can be raised to between 34-39° C. and the pH raised to about 7.2-7.4. These conditions can be imposed on the sperm by a first staining step where a first medium is introduced having the desired pH, osmolarty, and concentration of DNA selective dye. As but one example, this first medium can be a TALP based, or HEPES based solution supplemented with BSA (Bovine Serum Albumin), egg yolk, antibiotics and other additives.

In some embodiments, the sperm can be stained with a quenching dye, such as a red food dye, propidium iodide, or another dye with quenching properties. Conventionally, a second medium is prepared similar in composition to the first medium, but with the addition of a quenching dye and at a reduced pH in order to bring the pH of the overall sample back to a less damaging level for sperm. One embodiment of the present invention relates to a method of providing the second dye in a second medium at the same, or at a similar, elevated pH as the first medium.

One aspect relates to staining sperm with an improved quenching dye. This method can begin with the steps of obtaining sperm. The sperm can be obtained from fresh ejaculate, neat ejaculate or even thawed previously frozen ejaculate. The sperm can then be incubated with a fluorochrome dye under controlled staining conditions. The controlled staining conditions can include incubating at a temperature between 30 and 39° C., at a pH between 7.0 and 7.4 and at a time between 20 minutes and an hour. The fluorochrome dye can be a fluorescent dye such as Hoechst 33342. A quenching dye can be added to the sperm either after the step of incubation or during incubation. The quenching dye can be yellow food dye, orange food dye, green food due, or even blue food dye. More specifically, the quenching dye can be yellow food dye No. 6. Whichever, quenching dye is selected should be chosen for demonstrating improved resolution in sorting application such as microfluidic sorting or flow cytometry.

In one embodiment, the stained sperm can then be flowed through a sorter, such as a flow cytometer or microfluidic device, and exposed to radiant energy. Where a fluorescent dye, such as Hoechst 33342, is used, the radiant energy source can be a laser operated at the UV wavelength. This laser excitation can be used to distinguish between sperm having an X-chromosome and Y-chromosome based upon the energy fluoresced from the stained sperm.

In many embodiments, the stained sperm can be individually evaluated using flow cytometry, or another analytical technique based on fluorescent or visible light emissions. In flow cytometry, sperm are entrained within a fluid stream which is then broken off as droplets, each droplet ideally containing a single sperm which is individually irradiated with an energy source, such as a laser, at an inspection zone. A laser is one example of an energy source, but arc lamps and other sources of radiant energy can be used for irradiating the stained sperm. The DNA selective dye will absorb energy from the laser and emit light at a different wavelength in response to the excitation. The amount of this emission can be quantified to determine the relative amount of DNA selective dye compared to other sperm in the sample. The amount of the DNA selective dye can then be used to determine a characteristic of the sperm, and more particularly can be used to determine if individual sperm contains an X-chromosome or Y-chromosome.

A system for sorting sperm can include a sensor positioned to detect the interaction of the radiant energy with the DNA selective dye associated with the individual sperm at the inspection zone. The sensor, which can be a photomultiplier tube, can produce a signal based on the levels of these emissions, and can communicate to an analyzer for processing the signals and make sorting determinations on each event. The signal can be evaluated for evaluating DNA characteristics in individual sperm in the sample. DNA characteristics can include the presence of an X-chromosome or a Y-chromosome in individual sperm nuclei. Once a determination is made by the analyzer, a signal can be passed to a separator for separating the sample into distinct populations.

For example, in some sperm sorting systems using flow cytometry, sperm can be separated by electrically charging the stream entraining the sperm based upon the signal produced by the analyzer. The charged droplets that form and depart from the fluid stream then retain that charge and can be electromagnetically deflected by deflection plates guiding the droplet into one of several containers. Separated sperm can then be sorted into a plurality of collection elements depending on their DNA characteristics.

### DNA Fragmentation in Sorted Sperm.

Four experiments provided herein demonstrate sorting techniques that separate dead and DNA damaged sperm from a viable sperm sample. The sperm in the dead and dying sperm population present a higher frequency of DNA fragmentation, while the sperm separated into the viable subpopulation presents reduced levels of sperm DNA fragmentation. High levels of sperm damage can be detected by incorporating various sorting techniques, such as sex sorting using flow cytometry whereby the sperm can be sorted into a dead subpopulation while another fraction may contain a live subpopulation, as for example live X- and/or live Y-chromosome bearing subpopulations. Both the dead and the live subpopulations may contain cells that are damaged or are undergoing DNA fragmentation, but the proportional distribution will be minimal in the live subpopulations of cells.

### Experiments Conducted to Demonstrate the Decrease in DNA Damage.

In the following experiments, 5 Jersey and 15 Holstein bulls were selected between the ages of 3 and 9 years of age for sperm samples. Each sex sorted sample was sorted using a MoFlo SX TM (Beckman Coulter, Miami, Fla.). Sperm were sorted based on the difference of fluorescence signals generated using Hoechst 33342 (Molecular Probes, Eugene, Oreg.) and red food dye (FD&C#40, Molecular Probes Eugene, Oreg.). The resulting fluorescent signal was stronger for the cells having a higher DNA content, i.e. X-chromosome bearing populations versus those with lower DNA content, namely Y-chromosome bearing populations. The red food dye is typically excluded from those sperm having healthy intact membranes, while it is retained in sperm with damaged membranes quenching a significant amount of the fluorescence signal in those cells.

In each experiment, X- and Y-chromosome bearing, sorted sperm were selected based on differences in fluorescence signals using 16.2 mM Hoechst 33342 (Molecular Probes, Eugene, Oreg., USA), diluted in catch fluid consisting of a 20% egg yolk—TRIS extender. The same standards for routine semen preparation and cut-off values for standard semen characteristics for selecting the ejaculates for processing were applied. In all experiments, the bull ejaculates for processing either conventional or sex-sorted straws of semen were used only if they met the following criteria: 1) minimum motility of 55%; 2) minimum concentration of 900×10⁶ sperm/mL, as determined using the SP1-Cassette, Reagent S100, and NucleoCounter® SP100™ system (ChemoMetec A/S, Gydevang 43, DK-3450 Allerod, Denmark), but other comparable systems may be used; and 3) primary morphologies 15%, secondary morphologies 15%, and a total morphology count not to exceed 25%. Further, samples used in the post-thaw analyses had to meet standard quality control conditions of: 1) progressive motility of at least 45% at 0 h and 30% at 3 h; and 2) including intact acrosomes of at least 50% at 3 h. For three hour post-thaw motility and acrosome measurements, all samples were incubated for 3 h at 37° C. in a humidified chamber. For all semen quality evaluations, 75×25 mm glass microscope slides (Andwin, Addison, Ill., USA) and 22×22 mm #1.5 coverslips (Thomas Scientific, Swedesboro, N.J., USA) were used. All motility assessments were made using bright-field microscopy, and post intact acrosomes and morphology assessments were made using differential interference contrast (DIC) microscopy with a magnification of ×400.

All extenders used in the experiments were of the same formulation having a pH of 6.8 and an osmolarity balanced at 300 mOsm for the TRIS extender. For cryopreservation, sorted and conventional sperm samples were processed using a two step extension with glycerol. All frozen-thawed sex-sorted samples used in the experiments contained 2.1×10⁶ spermatozoa/straw (0.25 cc) while conventional samples had in the range of 25×10⁶ to 30×10⁶ spermatozoa/straw (0.5 cc).

Neat semen from each individual bull was divided into two aliquots. One aliquot was sex-sorted and thereafter the spermatozoa were frozen following cryostabilization using an automated freezing device, such as the IMV Digitcool® (IMV, Cedex, France) and stored in liquid nitrogen. The second aliquot was directly cryopreserved for subsequent analysis of the level of DNA fragmentation after thawing. The sperm DNA fragmentation analysis was performed on different subpopulations after X- and Y-chromosome sex selection, while comparing both aliquots for each respective bull.

In each experiment, DNA fragmentation was determined using a Sperm-Halomax kit (Halotech DNA, Madrid, Spain). Each sperm sample was lysed, then prepared in agarose on slides. The slides were then stained with SYBR I (Invitrogen, Molecular Probes, Eugene, Oreg.) or GELRED (Biotium, Hayward, Calif.) for staining chromatin which disperses differently around lysed sperm with DNA fragmentation and those without. Cells having fragmented DNA and those with intact DNA can then be visually distinguished on each slide.

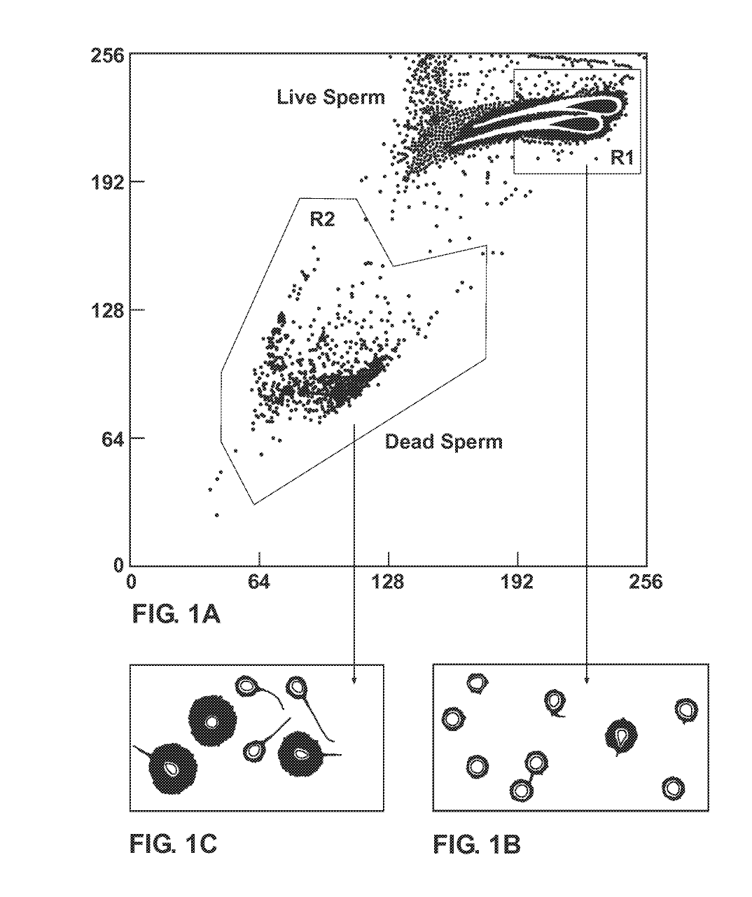

Referring to FIG. 1, a plot can be seen for sperm sorted in a flow cytometer of forward fluorescence versus side fluorescence (FIG. 1A). One subpopulation on this plot comprises a large proportion of spermatozoa which were dead or dying (R2 in FIG. 1A), and the other group comprises live spermatozoa (R1 in FIG. 1A). The regions for the sorting parameters as indicated on commercial flow cytometers such as the MoFlo SX or the MoFlo SX XDP (Beckman Coulter, Miami, Fla.) are indicated which illustrate plots for gating sperm cells based on forward and side fluorescence. FIG. 1B illustrates an in situ fluorescent micrograph of sperm cells from R1 using the Sperm-Halomax kit, in which the cells having small tight halos indicate sperm which had not undergone DNA fragmentation. In this slide, a single sperm can be seen with chromatin loosely spread around the membrane indicating this sperm had or was undergoing DNA fragmentation. Referring to FIG. 1C, sperm from R2 are illustrated, several of which can be seen with wide halos of dispersed chromatin indicating DNA fragmentation.

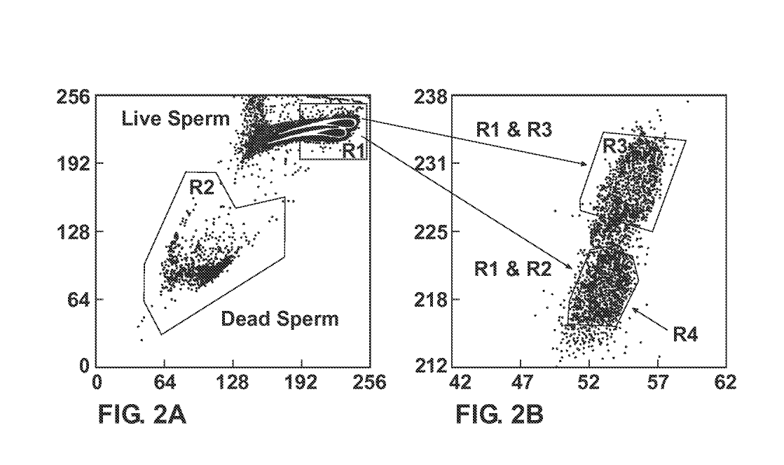

Referring to FIG. 2A and 2B, one subpopulation included spermatozoa which were predominantly dead (R2 in FIG. 2A) and the other two groups consisted of predominantly live spermatozoa (R1 in FIG. 2A) subpopulations containing X-chromosome bearing (R3 in FIG. 2B) and Y-chromosome bearing spermatozoa (R4 in FIG. 2B) at a purity of about 95%. The MoFlo SX XDP can be configured for gating each of R3, R4, and R2 into separate containers, while the MoFlo SX can be used for separating R1 sperm from R2 sperm. Sex ratio purities of the samples were determined using an STS Sexed Semen Purity Analyzer (Sexing Technologies, Navasota, Tex.), which provides high resolution peaks of X and Y chromosome bearing spermatozoa populations and basing each analysis on 2,000 spermatozoa. All of these subpopulations were analyzed and compared for the level of DNA fragmentation relative to the level obtained in the respective pre-sort sample taken after staining and incubation but before sorting. A total of 2×10⁶ spermatozoa for each sample were sorted. Dead spermatozoa were sorted based on Region 2 in FIG. 2A, excluding all other cells falling outside that region. The proportion of dead cells in the pre-sort semen samples averaged 13%, thereby providing an average sort speed of 800 to 900 dead spermatozoa per second. Therefore, about 85% of sperm containing fragmented DNA were removed by this process from the original sample.

### EXAMPLE 1

The first experiment was conducted to analyze the differences in the amount of DNA fragmentation before and after sex sorting. Sperm samples were taken from 5 jersey bulls and divided into two aliquots each. The first group of aliquots was sex sorted and cryopreserved. The second group of aliquots was directly cryopreserved. Table 1 illustrates the relative levels of DNA fragmentation obtained in each bull pre- and post-sex sorting.

TABLE 1
(% DNA Fragmentation, Sex Sorted)

| Reference | Pre-sort | Sort - XY |
|---|---|---|
| Bull 1 | 7.00 | 1.10 |
| Bull 2 | 7.50 | 1.10 |
| Bull 3 | 11.00 | 4.00 |
| Bull 4 | 9.00 | 5.00 |
| Bull 5 | 5.30 | 4.60 |
| Average ± SD | 7.96 ± 2.15 | 3.16 ± 1.91 |

The baseline level of DNA damage in the 5 presorted bull samples ranged from 5.3% to 11% with a mean and standard deviation of 7.9±2.1. The level of sperm DNA fragmentation obtained in sex sorted sperm samples was much lower, with a mean and standard deviation of 3.1±1.9. On average the reduction in sperm DNA fragmentation was 63%, but the reduction was as high as 85% in Bull 2.

### EXAMPLE 2

The second experiment specifically looked at the DNA fragmentation among each sorted subpopulation after sex sorting. Again 5 jersey bulls were used for this experiment; each was collected and sorted resulting in three subpopulations of sperm. The first subpopulation of sperm primarily consisted of those sperm considered dead via conventional sorting techniques as indicated by red food dye or propidium iodine. The second and third subpopulations primarily consisted of the live sorted sperm cells. A portion of each sample was
also tested prior to sorting in order to establish a baseline for DNA fragmentation. As shown in Table 2, the baseline for DNA fragmentation had a mean and standard deviation of 7.9±2.5. The DNA fragmentation determined in the sorted X-chromosome bearing subpopulation had a mean and standard deviation of 1.8±1.5, while the Y-chromosome bearing subpopulation had mean and standard deviation of 1.2±0.6 when averaged over each bull. The third subpopulation of sperm containing all the dead sperm tended to accumulate the majority of DNA fragmented sperm having a mean and standard deviation of 12±4.4.

TABLE 2
(% DNA fragmentation, sorted subpopulations)

| Reference | Pre - sort | Sort - Dead | Sort - X | Sort - Y |
|---|---|---|---|---|
| Average ± SD | 7.9 ± 2.5 | 12 ± 4.4 | 1.8 ± 1.5 | 1.2 ± 0.6 |

### EXAMPLE 3

The third experiment was conducted to analyze the distribution of sperm DNA fragmentation in 100 sex sorted straws after thawing, for comparing variations among samples taken at different times for 10 Holstein bulls. Each straw was collected and sex sorted for X-chromosome bearing sperm. Straws collected from the same bulls on different dates tended to present very similar DNA fragmentation, as can be seen in Table 3. While there were occasional outliers, the majority of samples taken from individual bulls demonstrated similar DNA fragmentation regardless of whether they were taken on different days.

TABLE 3
(% DNA Fragmentation, X-sorted samples taken different days)
Semen Sample

| Ref. | 1 | 2 | 3 | 4 | 5 | 6 | 7 | 8 | 9 | 10 | Avg. |
|---|---|---|---|---|---|---|---|---|---|---|---|
| HO-01 | 1.00 | 0.66 | 1.00 | 0.66 | 0.66 | 0.00 | 0.33 | 1.66 | 0.66 | 0.66 | 0.73 |
| HO-02 | 0.00 | 0.00 | 0.66 | 0.33 | 0.00 | 0.30 | 0.00 | 0.66 | 0.66 | 0.50 | 0.31 |
| HO-03 | 1.00 | 0.66 | 0.33 | 0.66 | 1.00 | 1.00 | 1.33 | 0.33 | 1.00 | 1.00 | 0.83 |
| HO-04 | 0.66 | 0.33 | 0.33 | 0.33 | 0.66 | 1.00 | 0.66 | 0.66 | 0.66 | 0.00 | 0.53 |
| HO-05 | 0.33 | 0.00 | 0.30 | 0.66 | 0.00 | 2.00 | 0.30 | 0.00 | 0.33 | 1.00 | 0.49 |
| HO-06 | 0.00 | 0.00 | 0.00 | 0.00 | 0.33 | 0.00 | 0.00 | 0.00 | 0.00 | 0.00 | 0.03 |
| HO-07 | 0.00 | 0.66 | 0.33 | 0.33 | 0.66 | 0.00 | 0.00 | 0.00 | 0.00 | 0.66 | 0.26 |
| HO-08 | 0.00 | 0.00 | 0.00 | 0.00 | 0.00 | 0.00 | 0.33 | 0.00 | 0.00 | 0.00 | 0.03 |
| HO-09 | 1.66 | 0.00 | 0.33 | 1.66 | 0.33 | 0.00 | 0.00 | 0.00 | 0.00 | 0.00 | 0.40 |
| HO-10 | 0.33 | 0.66 | 0.00 | 1.66 | 0.33 | 0.33 | 0.00 | 1.00 | 0.00 | 0.00 | 0.43 |

### EXAMPLE 4

The fourth experiment focused on evaluating DNA fragmentation in conventional and sorted samples at regular intervals in order to determine the rates at which DNA fragmentation occurs in each sample. Conventional sperm has been shown in previous experiments, and is shown again in Table 4, to have a higher baseline of DNA fragmentation as compared to sex sorted sperm. However, after monitoring sex sorted sperm at 24, 48 and 72 hours it appears sex sorted sperm is subject to a sharp increase in DNA fragmentation between about 24 and 48 hours, whereas conventional sperm maintain a baseline level until at least about 72 hours. At about 48 hours conventional sperm begin to exhibit slight increases in DNA fragmentation. Table 4 illustrates DNA fragmentation in eight bulls for conventional sperm at t0 (C-To), as well as sperm DNA fragmentation determined in sex sorted samples of the same bulls at a t0 (S-T0). As expected, S-T0 is categorically lower for each bull compared to C-T0. However, 24 hours later (S-T24), 48 hours later (S-T48) and 72 hours later (S-T72) the DNA fragmentation of the sex sorted samples change drastically, while conventional sperm tends to remain closer to its baseline level for about 72 hours. Table 4 also indicates a crossover positioning time point (CPT) which can be used as an indicator of the rate of sperm DNA fragmentation, for example, a lower CPT indicates a faster increase in DNA fragmentation and a higher CPT indicates a slower increase in DNA fragmentation. Averaged across each bull, the CPT for all 8 bulls averaged to about 33 hours. On average, after 33 hours the DNA fragmentation became greater in the sex sorted samples than in the conventional samples.

TABLE 4
(CPT and % DNA Fragmentation - Sorted Over Time)

| | CPT | C-T0 | S-T0 | S-T24 | S-T48 | S-T72 |
|---|---|---|---|---|---|---|
| Bull 1 | 27 h | 5.00 | 0.33 | 1.66 | 10.50 | 70.00 |
| Bull 2 | 44 h | 2.00 | 0.33 | 0.66 | 5.00 | 45.00 |
| Bull 3 | 25 h | 2.66 | 0.33 | 0.66 | 17.00 | 32.00 |
| Bull 4 | 33 h | 2.00 | 0.33 | 0.00 | 11.00 | 26.00 |
| Bull 5 | 51 h | 4.00 | 0.00 | 0.66 | 2.50 | 66.00 |
| Bull 6 | 41 h | 4.00 | 2.00 | 2.66 | 8.00 | 29.00 |
| Bull 7 | 27 h | 3.33 | 0.00 | 1.00 | 19.66 | 32.00 |
| Bull 8 | 19 h | 1.00 | 0.00 | 1.00 | 19.00 | 37.00 |
| r2 | | 0.31 | 0.27 | 0.59 | 0.64 | 0.68 |
| Durbin- | | 2.05 | 1.98 | 1.99 | 2.41 | 2.55 |
| Watson | | | | | | |
| P | | 0.24 | 0.26 | 0.27 | 0.09 | 0.06 |

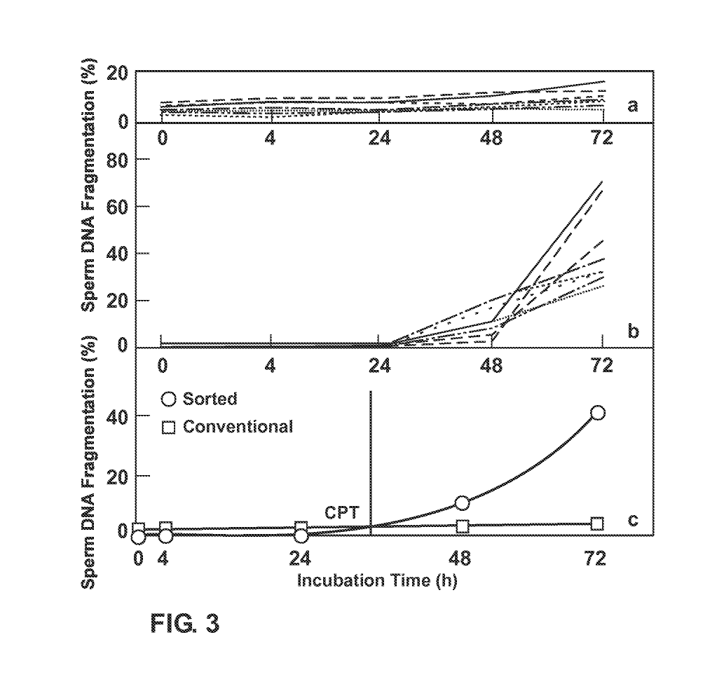

FIG. 3 illustrates a graphical representation of the data in Table 4. FIG. 3A illustrates the percentage of DNA fragmentation over time for about 72 hours. In comparison, FIG. 3B illustrates the DNA fragmentation in sorted sperm over the course of 72 hours. Contrasting FIG. 3A with FIG. 3B it can be seen the conventional sperm increases at a slower and more steady rate over 72 hours of incubation, while the sorted sperm often presents sharp increases in sperm DNA fragmentation between about 24 and 48 hours. FIG. 3C illustrates the mean the conventional samples and the mean of the sorted samples and by doing so more clearly demonstrates the sorted sperm having lower percentages of DNA fragmentation initially. FIG. 3C also more clearly illustrates the sharp increase DNA fragmentation among the sorted sperm relative to the conventional sperm and the CPT, where on average the sorted sperm began presenting more DNA fragmentation than the sorted sperm. For the samples taken and illustrated in FIG. 3C the CPT occurs around 33 hours. Therefore one objective of the embodiments presented herein is extending the CPT time of sex sorted sperm in order to increase the useful life of sex sorted sperm.

Reducing Sperm DNA Fragmentation with Modified Processes Including Quinolones

### EXAMPLE 5

The fifth experiment illustrates a connection between the degradation of sperm DNA and the presence of a bacterial infection (BI). This experiment shows bacterial infections present after thawing semen in cases where bacteria were not initially detected and even when the semen was cryopreserved in conventional extenders with antibiotics. The first experiment also illustrates the rate of sperm DNA fragmentation in samples with bacterial infections tends increase very quickly in logarithmic manner, while DNA fragmentation in samples without bacterial infections tends to increase more slowly and in a linear fashion. This experiment further illustrates a relationship between the sperm DNA fragmentation and the bacterial load.

Commercially cryopreserved semen samples from 47 different Holstein bulls were included in the analysis. Six straws were randomly selected from each bull with the criterion being straws from different ejaculates. A total of 282 straws were assessed for the dynamics of sperm DNA fragmentation. All animals were aged 13-125 months, healthy and under controlled feeding, housing, and photoperiod conditions. Semen samples were collected using an artificial vagina and following quality control of standard semen characteristics, each ejaculate was divided into different single doses using the commercial extender INRA 96 medium containing egg yolk (IMV Technologies, Spain) and frozen using an automated freezing device, IMV Digitcool (IMV, Cedex, France) and stored under liquid nitrogen. The threshold level to consider that a sample was infected with bacteria was established as five bacteria per microscope field when analyzed under a 40× objective.

Cryopreserved samples were thawed by immersion in a 37° C. water bath for 30 seconds. Straws were incubated in a 37° C. water bath for up to 4 days. Determination of sperm DNA fragmentation was conducted after 0, 4, 24, 48, 72 and 96 hours of incubation. Each straw was diluted to 5-10×10⁶ spermatozoa/mL in INRA 96 medium (IMV Technologies, Spain), and sperm DNA fragmentation was tested by the Sperm-Halomax® kit (Halotech DNA, Spain). To perform each experiment 25 μL of each diluted aliquot were used. This volume was mixed with 50 μL of low melting point agarose. Ten μL of the mixture were extended upon pre-treated slides (Sperm-Halomax® kit), covered with a 23×23 mm coverslip and placed on a cold metallic plate in the fridge (4° C.) for 5 minutes. Afterwards, the coverslip was removed and each slide was set up horizontally in 10 mL of lysing solution (Sperm-Halomax® kit) for 5 minutes and the slide was washed in dH2O for 5 min at room temperature. The nucleoids resulting from the lysing process were dehydrated in a 2 minute series of ethanol baths (70%, 90% and 100%). Once dried, the slides were stained using a 1/1 proportion of 10× Gel Red fluorocrome (Biotium, USA) and Vectashield anti-fading medium (Vectashield, Vector, Burlinghan, Calif., USA). Samples were visualized with fluorescence microscopy and green excitation. Spermatozoa were counted and divided into two groups: fragmented and non-fragmented, and a percentage was calculated based on measuring 300 sperm. Sperm samples from six different individuals were studied to determine the bacterial phyla present in Holstein bull semen samples. In three of the samples, infection was not detected using fluorescence microscopy, while infection was clear in the other three individuals even when the samples were assessed for BI after thawing. DNA extraction of the samples using phenol-chlorophorm isoamil alcohol (25:24:1) (Amresco Inc.) and Cetyl trimethylammonium bromide (CTAB) (Sigma-Aldrich S.A.). DNA amplification was done via Polymerase Chain Reaction (PCR) using primers designed for specific regions of the bacterial gene that codifies for ARNr 16S. Primer sequences are: Forward 5′-GAG TTT G(AC)T CCT GGC TCA G-3′, and Reverse 5′-ACG G(CT)T ACC TTG TTA CGA CTT-3′. DNA purification was performed using Illustra GFX PCR DNA and Ged Band Purification kit (General Electric Healthcare S.A.). Ligation of bacterial DNA fragments to the clonation vector pGEM®-T easy (Promega S.L) and transformation of JM109 competent cells was performed using the pGEM®-T easy vector systems kit following the pGEM®-T easy vector system protocol.

Ten white colonies were selected from each semen sample and DNA extraction was carried out. Finally, plasmid DNA extracted from the 60 colonies was sequenced in the forward direction.

The statistical analysis was performed using SPSS version 17.0. Data base Ribosomal database Project (http://rdp.cme.msu.edu/classifier/classifier.jsp) was used to relate sequences of rRNA 16S with a specific bacterial taxon.

High resolution microscope images were obtained when GelRed staining was combined with the sperm chromatin dispersion test. Sperm heads presenting haloes of dispersed chromatin spots contain fragmented DNA, while heads with small haloes are indicative of sperm without DNA fragmentation.

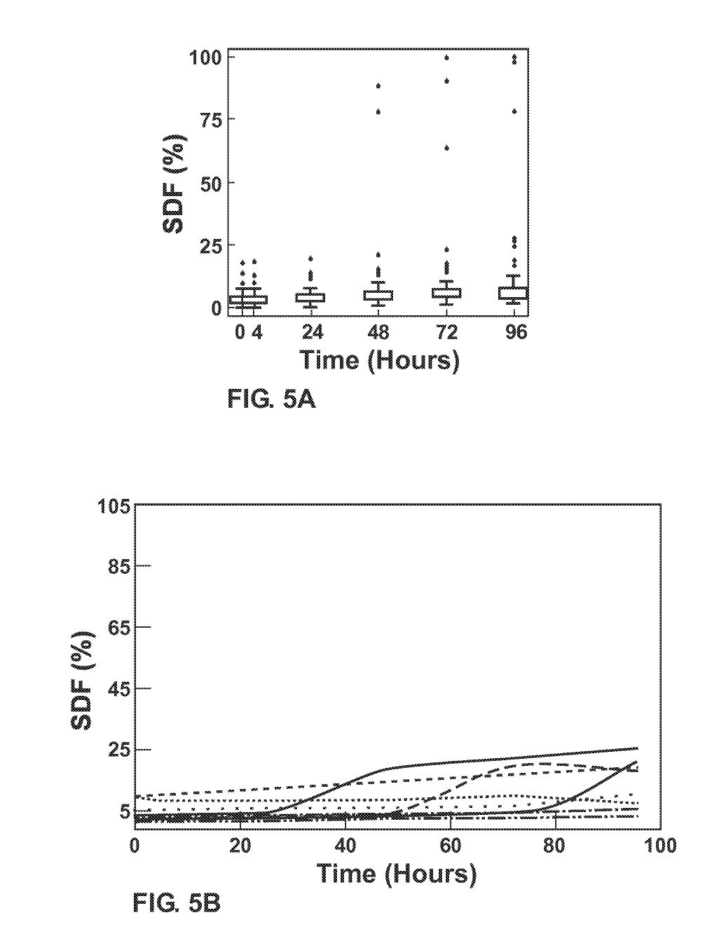

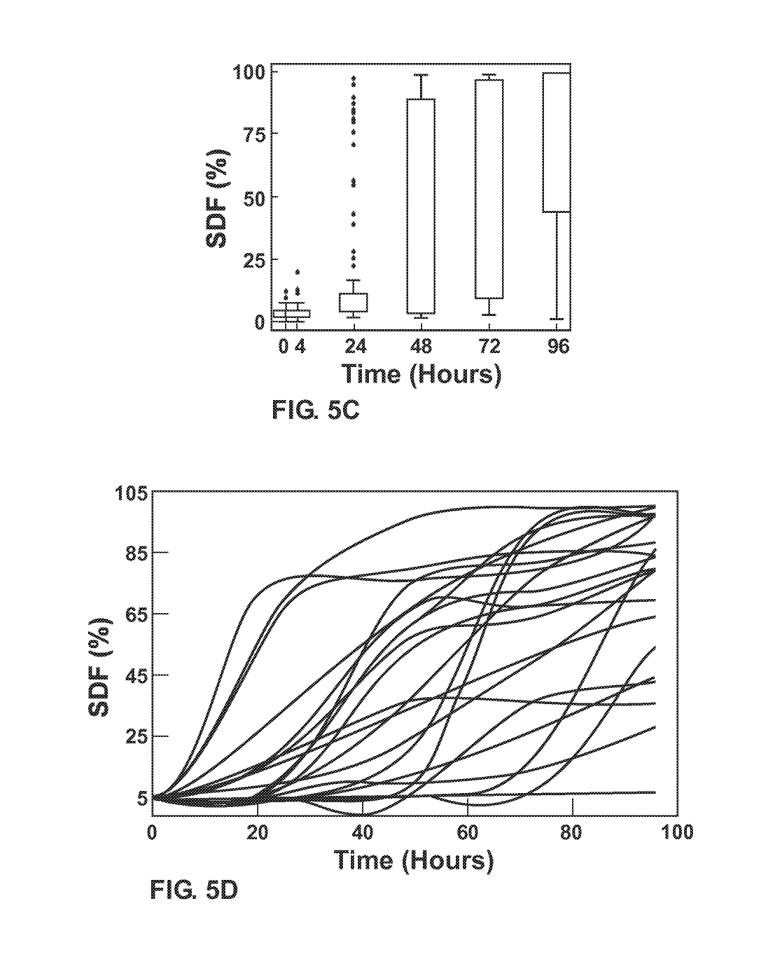

The baseline level (T0) of sperm DNA fragmentation was very similar in all bulls presenting values never over 20% and with an average of 3.65%±1.55%. However, after incubation at 37° C., the level of sperm DNA fragmentation (SDF) increased over time in each semen sample. To analyze the influence of bacterial infection on sperm quality over time, the sperm DNA fragmentation was assessed at different times of incubation and the data plotted and compared according to the cluster criteria “animals with bacterial infection versus animals free of infection.” The values for sperm DNA fragmentation at different intervals are illustrated for the groups without bacterial infections FIG. 5A as well as the groups where a bacterial infection was present FIG. 5B. A dynamic graphic representation of non-infected FIG. 5C, and infected semen samples FIG. 5D illustrates the several differences in the rate of sperm DNA fragmentation in the presence of a bacterial infection.

After analyzing six different straws of each bull in 47 individual Holstein bulls, it was observed that all straws from 24 bulls were free of infection after 4 days of incubation at 37° C., while bacterial infection was detected in 23 of them at different times of incubation (Table 5).

TABLE 5
(Percentage of infected straws and time of infection detection)

| | T0 | T4 | T24 | T48 | T72 | T96 |
|---|---|---|---|---|---|---|
| H-1 | | | 84% | 100% | | |
| H-2 | | | 84% | | | 100% |
| H-3 | | | 67% | 84% | | 100% |
| H-4 | | | | 67% | 84% | 100% |
| H-5 | | | 50% | 84% | | 100% |
| H-6 | | | 33% | 84% | | 100% |
| H-7 | | | 33% | 100% | | |
| H-8 | | | 33% | 84% | | 100% |
| H-9 | | | 50% | 84% | 100% | |
| H-10 | | 17% | 50% | 84% | | 100% |
| H-11 | | | | | | 100% |
| H-12 | 50% | | | 84% | | 100% |
| H-13 | | | 67% | 84% | 100% | |
| H-14 | 84% | | | | 100% | |
| H-15 | 67% | | 84% | 100% | | |
| H-16 | | | 33% | | | 100% |
| H-17 | 100% | | | | | |
| H-18 | | | 84% | | 100% | |
| H-19 | | | 17% | 67% | 84% | 100% |
| H-20 | | | 33% | 50% | 67% | 100% |
| H-21 | | | 33% | 67% | 100% | |
| H-22 | 17% | | 33% | | 67% | 100% |
| H-23 | 33% | | | | | 100% |

Positive presence of bacteria at different incubation times, according to the threshold level established is shown in Table 5. Differences among bulls were observed for the time when bacterial presence was detected. In some cases (H-11, H-14, H-17, H-22 and H-23) the infection was detected right after thawing; however, in other cases the presence of infection was delayed from 24-48 h. The proportion of infected straws at a specific incubation time also varied within each individual (Table 5). Nevertheless, in general, the majority of the straws from the same bull exhibited detectable infection at the same time period after incubation. Of those 23 bulls presenting bacterial infection, the infection was positively detected in 15% of the straws at T0, in 50% of the straws after 24 hours of incubation, and in all straws after 96 hours of incubation.

To analyze the relative rate of SDF (rSDF), the slopes of each regression line at different intervals were compared. The rSDF was expressed as the increase of Sperm DNA Fragmentation per hour. The whole incubation time (T0-T96) was divided into two intervals: from T0 to T48 and from T48 to T96. The rSDF was calculated for each time interval with results shown in Table 6. The rate of DNA damage was higher in those semen samples having bacterial infection (Table 6a). On average, the whole rSDF estimated for infected samples was 0.7 per hour, while those samples free of infection the SDF rate was 0.05 per hour Table 6b). Interestingly, some infected straws did not exhibit pronounced slopes for the dynamic of DNA damage and they behave as straws free of infection, as represented by the curves close to the X axis in FIG. 5D.

TABLE 6a
(Rate of sperm DNA fragmentation in different
bulls showing Bacterial infection)

| | T0-T48 | T48-T96 | T0-T96 |
|---|---|---|---|
| I1 | 1.92 | 0.09 | 1.01 |
| I2 | 1.57 | 0.12 | 0.84 |
| I3 | 1.24 | 0.41 | 0.82 |
| I4 | 0.06 | 0.79 | 0.42 |
| I5 | 1.03 | 0.35 | 0.69 |
| I6 | 1.22 | 0.48 | 0.85 |
| I7 | 1.10 | 0.81 | 0.96 |
| I8 | 1.29 | 0.29 | 0.79 |
| I9 | 1.47 | 0.33 | 0.90 |
| I10 | 1.04 | 0.49 | 0.77 |
| I11 | 0.02 | 0.02 | 0.02 |
| I12 | 0.08 | 1.72 | 0.90 |
| I13 | 0.35 | 1.64 | 0.99 |
| I14 | 0.66 | 0.64 | 0.65 |
| I15 | 0.02 | 1.99 | 1.01 |
| I16 | 0.67 | 0.00 | 0.33 |
| I17 | 1.28 | 0.70 | 0.99 |
| I18 | 1.54 | 0.44 | 0.99 |
| I19 | 0.38 | 1.18 | 0.78 |
| I20 | 0.05 | 0.81 | 0.43 |
| I21 | 0.63 | 1.29 | 0.96 |
| I22 | 0.03 | 1.06 | 0.55 |
| I23 | 0.10 | 0.39 | 0.25 |
| Mean | 0.77 | 0.70 | 0.73 |

TABLE 6b
(Rate of sperm DNA fragmentation (rSDF) in different bulls showing
absence of Bacterial infection)

| | T0-T48 | T48-T96 | T0-T96 |
|---|---|---|---|
| I24 | 0.35 | 0.16 | 0.25 |
| I25 | 0.04 | 0.07 | 0.05 |
| I26 | 0.02 | 0.02 | 0.02 |
| I27 | 0.01 | 0.31 | 0.16 |
| I28 | 0.03 | 0.05 | 0.04 |
| I29 | 0.02 | 0.35 | 0.19 |
| I30 | 0.02 | 0.01 | 0.01 |
| I31 | 0.10 | 0.11 | 0.10 |
| I32 | 0.02 | 0.02 | 0.02 |
| I33 | 0.02 | 0.00 | 0.01 |
| I34 | 0.08 | 0.12 | 0.10 |
| I35 | 0.03 | −0.01 | 0.01 |
| I36 | 0.03 | 0.03 | 0.03 |
| I37 | 0.05 | 0.05 | 0.05 |
| I38 | 0.01 | 0.01 | 0.01 |
| I39 | 0.02 | 0.02 | 0.02 |
| I40 | 0.02 | 0.02 | 0.02 |
| I41 | 0.01 | 0.00 | 0.00 |
| I42 | 0.02 | 0.06 | 0.04 |
| I43 | −0.01 | 0.00 | −0.01 |
| I44 | 0.02 | 0.03 | 0.02 |
| I45 | 0.02 | 0.03 | 0.03 |
| I46 | 0.01 | 0.01 | 0.01 |
| I47 | 0.02 | 0.03 | 0.03 |
| Mean | 0.04 | 0.06 | 0.05 |

Differences in the level of DNA damage were not observed at T0, when both cohorts of animals were compared (F=0.761; P=388). Comparing FIGS. 5A, 5B and FIGS. 5C and 5D levels of DNA damage rapidly changes. Thus, when the level of sperm DNA fragmentation in infected and non infected straws were compared at subsequent incubation times, the statistical values obtained were: T0h: F=0.425; P=0.388; T4h: F=0.425; P=0.518; T24h: F=6.895; P=0.012*; T48h: F=31.477; P=0.000*; T72h: F=73.255; P=0.000*; T96h: F=132.860; P=0.000* (*significant differences). Statistical analysis showed that bacterial infection, in general, significantly increased the level of sperm DNA fragmentation at 24 hours of incubation. However, in some cases (see FIG. 5D) a rapid increase in the level of sperm DNA fragmentation was observed during the first hours after incubation.

In addition, analyzing the dynamic distribution of SDF in each bull, three different basic distributions for the rSDF could be obtained: logarithmic, linear or exponential. A logarithmic function is concomitant with a high increase in SDF during the first hours of incubation, while an exponential function explains that SDF is increasing slowly during the first hours of incubation. Results for grouping the different animals according to the higher R² values obtained for each distribution are given in Table 7. While logarithmic curves were the main trend in contaminated samples, linear curves were the major trend in non-contaminated samples (Table 7). Additionally, the slope of the curve must also be taken into account because low slope values are better in terms of DNA fragmentation dynamics for similar R² values.

TABLE 7
(Distribution of the different curves for the rSDF
in all Holstein bulls studied)

| | Linear | Logarithmic | Exponential |
|---|---|---|---|
| Bulls with BI in sperm | 2 | 16 | 5 |
| Bulls with BI in sperm | 21 | 2 | 1 |

Table 8 illustrates that previously undetected bacterial loads can quickly degrade sperm DNA integrity. Whereas, the DNA fragmentation rate in sperm that ended up with bacterial infections tended to increase in a logarithmic fashion, sperm which did not present bacterial infection tended to have DNA fragmentation that increased linearly. It should be noted NRA 96 described with this example contains the antibiotics Penicillin and Gentamycin. Gentamycin is widely used in sperm suspensions, but can damage sperm membranes above certain concentrations. Therefore, the fact bacterial infections developed even in an extender containing these antibiotics illustrates the need for a less harmful sterile media.

### EXAMPLE 6

The sixth example illustrates the use of Ciprofloxacin, a quinolone from the fluoroquinolone subset, as an antibiotic used for the preservation of reproductive cells such as sperm. Ciprofloxacin (quinolone) treatments where shown in this experiment to reduce the sperm DNA fragmentation occurring due to bacterial infections.

This example utilized a total of four bulls. Two of the bulls were classified as bad bulls; in that prior to this experiment these bulls had demonstrated a high incidence of bacterial infections in their sperm samples after 24 hours. Such results can consistently occur in an otherwise healthy bull for a variety of reasons. Regardless of the exact cause, two bulls were selected for their historical production of semen samples with bacterial infections. Similarly, two bulls were selected which historically produced semen sample without bacterial infections.

Semen was collected from each of the four bulls and tested at collection, then again at the 24 and 48 hour marks. DNA fragmentation was determined with high resolution microscope images which were obtained when GelRed staining was combined with the sperm chromatin dispersion test. Sperm heads presenting haloes of dispersed chromatin spots contain fragmented DNA, while heads with small haloes are indicative of sperm without DNA fragmentation.

For each of the four bulls a control group was established, and a second group was treated with Ciprofloxacin at 1 μg/mL diluted in Acromax, stained at the time of thawing (Time 0 Hour). Table 8 shows the results for each of the control group and the treated group at the time of staining, at 24 hours, and at 48 hours.

TABLE 8
(Rate of sperm DNA fragmentation in bulls
treated with Ciprofloxacin and a control)

| | Time 0 Hour | | Time 24 Hour | | Time 48 Hour | |
|---|---|---|---|---|---|---|
| | With | | With | | With | |
| | Anti- | Without | Anti- | Without | Anti- | Without |
| Bull | biotic | Antibiotic | biotic | Antibiotic | biotic | Antibiotic |
| Bad I | 3.00% | 2.33% | 2.33% | 12.67% | 2.00% | 24.33% |
| Bad II | 3.00% | 4.67% | 3.00% | 12.33% | 3.00% | 80.00% |
| Good I | 3.33% | 2.33% | 3.33% | 3.67% | 2.33% | 3.67% |
| Good II | 2.67% | 2.67% | 4.00% | 3.00% | 3.67% | 4.00% |

Table 8 illustrates a relatively consistent 2-3% sperm DNA fragmentation in both bad bull I and bad bull II for the sperm treated with Ciprofloxacin, indicating no bacterial infections in these samples In sharp contrast, the control for bad bull 1 escalates to 12.67% sperm DNA fragmentation after 24 hours, then to 24.33% DNA sperm fragmentation. The sharp rise in DNA fragmentation is indicative of bacterial infections in these samples. Bad bull 2 samples may start with a higher bacterial load, because the sperm DNA fragmentation increases very quickly, from 4.67% to 12.33% at the 24 hour mark, then up to 80% at the 48 hour mark, indicating significant bacterial infections in the untreated bad bull II samples.

Each of the treated and untreated samples from good bull I and II maintain relatively consistent sperm DNA fragmentation between about 3-4% from the time of collection up through 48 hours. These results indicate both that Ciprofloxacin is effective in preventing bacterial infections, and that the use of Ciprofloxacin does not promote DNA fragmentation and sperm deterioration, as many antibiotics do at certain levels. These results also indicate a lack of bacterial infections in the samples from both good bull I and good bull II. The treated and untreated samples from good bull I and good bull II also indicate that the Ciprofloxacin treatment did not promote sperm DNA fragmentation, as the uninfected treated samples and uninfected untreated sample demonstrated similar levels of sperm DNA fragmentation.

Surprisingly, Ciprofloxacin at 1 μg/mL achieved good results in preventing bacterial infections and the accompany increase in DNA fragmentation. Antibiotics known in the art for preventing bacterial infections in sperm samples fail to prevent many infections because at the dosage required to kill all, or nearly all, the bacteria is harmful to the sperm membrane. Therefore, these antibiotics must be used in smaller dosages.

In one embodiment the present claimed invention relates to a sperm suspension formed for the purpose of preserving sperm for storage or processing, and specifically one including Ciprofloxacin (quinolones) for the purpose of eliminating, or nearly eliminating, bacterial infections and this improving sperm DNA fragmentation. In particular, certain embodiments of the claimed invention relate to a sperm suspension which can include any of many know sperm extenders known in the art. Typically, cell samples such as sperm samples are collected at very high natural concentrations. While these concentrations can vary from species to species, these samples tend to be much too highly concentrated for effective sorting or storage. The sperm samples can then be diluted with extenders for establishing useful concentrations of sperm. The extenders further provide mediums for keeping sperm healthy and motile for processes such as storage, fertilization, or sorting. By way of an illustrative example, extenders such as TRIS extenders, TALP extenders, and a HEPES INFA 39 can be used. These extenders can optionally include an antibacterial component, as well as agents for regulating oxidation uptake or reversibly reducing sperm motility.

### Reduction in Sperm DNA Fragmentation by Modifications to Sperm Staining Protocols

Flow cytometry technology for the sorting of X- and Y-chromosome bearing sperm is currently utilized in research and for commercial applications. During sample preparation previous to the sex-sorting process, sperm go through different pH treatments that might affect their quality. The differences in the pH of sperm extenders like those typically used for sperm sex-sorting could result in differing amounts of sperm DNA damage.

A key step in the sex sorting process is the staining of a sperm sample with a fluorescent dye, which is typically carried out with Hoechst 33342. In order for this fluorescent dye to permeate the sperm cell membrane and associate stoichiometrically with the sperm DNA the temperature and pH of the sperm sample must be elevated beyond the levels conducive to healthy sperm. Conventionally, the sperm pH was elevated to about 7.4 pH with a clear TALP at 7.4 pH. In order to reduce the staining time the sperm sample is then incubated with the dye at an elevated temperature between about 34° C. and 39° C.

Previously, the pH of the sperm sample was returned to normal pH ranges (e.g. 6.8 pH for bovine) by the addition of a second TALP after the Hoechst staining. The second TALP is generally similar to composition to the clear TALP, but can contain a red food dye and a pH of 5.5. This second TALP is often referred to as red TALP. The 5.5 pH was previously designed to bring the overall pH of the sample back to about sperms normal pH ranges (e.g. 6.8 pH for bovine).

In the following example, fresh semen samples from 30 bulls were checked for industry acceptable motility, concentration, and morphologies. Each bull provided two samples from different ejaculates. The pH of the samples used varied between 6.1 and 7.6 with an average of 6.6. Each sperm sample was stained with Hoechst 33342 and a calculated amount of TALP based on the ejaculate concentration, per industry standards for staining in conjunction with sorting in a flow cytometer. Each sample was subsequently mixed with a red TALP, which includes red food dye, at 5.5 pH, 6.4 pH and at 7.4 pH. The red TALP at 5.5 represents the industry standard for adding red food dye as part of the staining procedure.

Red TALP can be prepared with, for example, 4% egg yolk in a HEPES based medium including glucose and BSA, but those of ordinary skill in the art will appreciate other percentages of egg yolk can be prepared. NaOH or HCl can be added to red TALP in order to adjust the TALP to the desired pH.

### EXAMPLE 7

Fresh semen samples from different breeds of bulls (n=30) were obtained from one bull stud in Texas (Sexing Technologies, Navasota, Tex., USA). Two samples were randomly selected from each bull with the criteria “samples from different ejaculates”. The pH of the samples used in this study had an average of 6.6 with values between 6.1 and 7.6.

All animals were healthy and under controlled feeding, housing and received natural photoperiod conditions and ambient temperatures. Semen samples were collected using an artificial vagina and underwent the quality control of standard semen characteristics (minimum motility of ≧55%; minimum concentration of ˜900 million/mL, determined using the SP1-Cassette, Reagent S100 and NucleoCounter® SP-100™ system—ChemoMetec A/S, Gydevang 43, DK-3450 Allerod, Denmark—; and primary morphologies ≦15%, secondary morphologies ≦15%, and with a total morphology count that could not exceed 25%.).

Each ejaculate was divided into four separate doses and placed in 12×75 mm tubes (Hauppauge, N.Y.). One neat semen sample was kept as a control and the other three aliquots were treated with 16 μL of 8.1 mM Hoechst 33342 (Molecular Probes, Eugene, Oreg., USA) and a calculated amount of modified Tyrode's albumin lactate pyruvate (clear TALP) pH 7.4 based on neat ejaculate concentration. For the separation of live and dead sperm during the sex-sorting process, red TALP at three different pH treatment levels: 5.5, 6.4, and 7.4 was added to the sperm samples.

The dynamics of sperm DNA fragmentation were assessed an hour after adding the red TALP treatment (T0Hr) and at 24 hours of incubation at 34° C., using the SCDt (Fernández et al. 2005; López-Fernández et al. 2007), the bull Sperm-Halomax® kit (Halotech DNA, Madrid, Spain). The final concentration used for assessing sperm DNA damage was adjusted to 3-5×10⁶ sperm/mL with clear TALP pH 7.4.

To perform each experiment, 5 microliters (μL) of each diluted aliquot were mixed with 10 μL of low melting point agarose. Next, 2 μL of the mixture were extended upon 8 circle pre-treated slides (Sperm-Halomax® kit), covered with a 23×23 mm coverslip and placed on a cold metallic plate in the refrigerator (4° C.) for 5 minutes. Afterwards, the coverslip was removed and each slide was set up horizontally in 10 mL of lysing solution (Sperm-Halomax® kit) for 5 minutes and then washed in dH2O for 5 minutes at room temperature. The nucleoids resulting from the lysing process were dehydrated in a 2 minute series of ethanol baths (70%, 90% and 100%). Once dried, the slides were stained using a 1:1 proportion of Sybr Green® 10× fluorochrome (Biotium Inc., Hayward, Calif., USA) and Vectashield® (Vector Laboratories Inc. Burlingame, Calif., USA) Mounting Medium. Samples were visualized with fluorescence microscopy using a Leica DMLA model motorized epifluorescence microscope controlled with software for automatic scanning A Leica EL6000 fluorescence light source equipped with a metal halide lamp and Plan-Fluotar 40× objective for routine was employed.

Sperm heads presenting small and compact haloes of chromatin dispersion contain an orthodox DNA molecule, while heads presenting big and spotty haloes of dispersed chromatin identify sperm with fragmented DNA. Sperm were counted and divided into two groups: fragmented and non-fragmented, and a percentage was calculated based on measuring 300 sperm.

For a preliminary revision of the data, graphics were created using Microsoft Office Excel 2007. Analysis of Variance (ANOVA) was used to determine if there were statistical differences (α=0.05) among mean values of the groups (SPSS v.17.0 for Windows, SPSS Inc., Ill., USA). Bonferroni post-hoc tests were utilized to determine the pair wise directional differences between groups.

High resolution microscope images were obtained when Sybr Green staining was combined with the sperm chromatin dispersion test. Sperm heads presenting haloes of dispersed chromatin spots contain fragmented DNA, while heads with small haloes are indicative of sperm without DNA fragmentation.

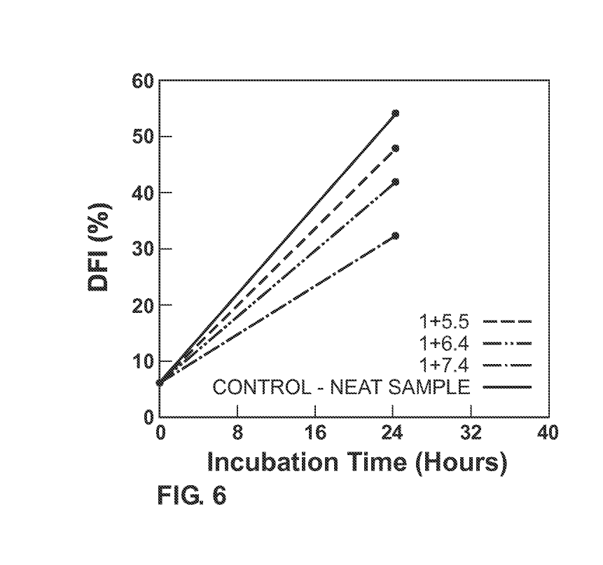

To analyze the influence of pH extender on sperm quality over time, the sperm DNA fragmentation was assessed at two different incubation times, 0 and 24 hours, and the data plotted and compared according to the pH of the extender. The values for sperm DNA fragmentation at different times and a dynamic graphic representation per treatment (average values for all bulls), show clear differences when the groups are plotted in FIG. 6.

The baseline level (T0) of sperm DNA fragmentation in the raw sample was very similar in all bulls, never presenting values over 18%, with an average of 6.5%±3.9%. This value did not show significant changes at 0 Hr (P=0.749; F=0.406) when adding the different pH treatments (pH 5.5: 5.7+3.9%; pH 6.4: 5.5+3.9%; pH7.4: 5.1+3.9). However, after sperm incubation for 24 Hr at 34° C., sperm DNA fragmentation increased differentially (P=0.001; F=5.640) over time according to the pH of the extender added.

Bonferroni post-hoc tests also determined that there are no significant differences on the DFI between pH groups at 0 Hr (Table 10). However, at 24 Hr, significant differences on DFI appear between the group of samples treated with red TALP 7.4 and the raw semen (P=0.001) as well as the ones treated with red TALP 5.5 (P=0.039) (Table 9).

TABLE 9
(Bonferroni post-hoc test for DFI for pH
treatments and neat-control at 9 hours)
Bonferroni

| | | | | | 95% Confidence | |
|---|---|---|---|---|---|---|
| | | | | | interval for Mean | |
| | | Mean | Std. | | Lower | Upper |
| pH 1 | pH 2 | difference | Error | Sig. | Bound | Bound |
| 0 | 5 | .741 | .779 | 1.000 | −1.36 | 2.84 |
| | 6 | 1.333 | .779 | .540 | −.76 | 3.43 |
| 5 | 7 | 1.259 | .779 | .655 | −.84 | 3.36 |
| | 0 | −.741 | .779 | 1.000 | −2.84 | 1.36 |
| | 6 | .593 | .779 | 1.000 | −1.50 | 2.69 |
| | 7 | .519 | .779 | 1.000 | −1.58 | 2.61 |
| 6 | 0 | −1.333 | .779 | .540 | −3.43 | .76 |
| | 5 | −.593 | .779 | 1.000 | −2.89 | 1.50 |
| | 7 | −.074 | .779 | 1.000 | −2.17 | 2.02 |
| 7 | 0 | −1.259 | .779 | .655 | −3.36 | .84 |
| | 5 | −.519 | .779 | 1.000 | −2.61 | 1.58 |
| | 6 | .074 | .779 | 1.000 | −2.02 | 2.17 |

Not significant differences are shown (p > 0.05).

TABLE 10
(Bonferroni post-hoc test for DFI for pH
treatments and neat-control at 24 hours)
Bonferroni

| | | | | | 95% Confidence | |
|---|---|---|---|---|---|---|
| | | | | | interval for Mean | |
| | | Mean | Std. | | Lower | Upper |
| pH 1 | pH 2 | difference | Error | Sig. | Bound | Bound |
| 0 | 5 | 6.688 | 5.794 | 1.000 | −8.85 | 22.22 |
| | 6 | 13.563 | 5.794 | .125 | −1.97 | 29.10 |
| | 7 | 22.750* | 5.794 | .001 | 7.22 | 38.28 |
| 5 | 0 | −6.688 | 5.794 | 1.000 | −22.22 | 8.85 |
| | 6 | 6.875 | 5.794 | 1.000 | −8.66 | 22.41 |
| | 7 | 16.063* | 5.794 | .039 | .53 | 31.60 |
| 6 | 0 | −13.563 | 5.794 | .125 | −29.10 | 1.97 |
| | 5 | −6.875 | 5.794 | 1.000 | −22.41 | 8.66 |
| | 7 | 9.188 | 5.794 | .692 | −6.35 | 24.72 |
| 7 | 0 | −22.750* | 5.794 | .001 | −38.28 | −7.22 |
| | 5 | −16.063* | 5.794 | .039 | −31.60 | −.53 |
| | 6 | −9.188 | 5.794 | .692 | −24.72 | 6.35 |

Significant differences on DFI appear between the group of samples treated with red TALP 7.4 and the raw semen (P=0.001) as well as the ones treated with red TALP 5.5 (P=0.039).

The results of this study demonstrate that the DNA molecule can be affected by pH fluctuations. It appears that lower pH could increase the rate at which DNA becomes damaged over time. This may be important in refining the sex-sorting process. Notably, undiluted neat semen controls had greater levels of DNA fragmentation overall, suggesting a negative effect of high sperm concentrations and seminal plasma.

### Improved Processes for Reducing DNA Fragmentation in Sorted Sperm.

In one embodiment, sperm is processed with a quinolone in the fluoroquinolone subset; namely ciprofloxacin. This antibiotic demonstrated an ability to kill all, or nearly all, the bacteria which survived conventional antibiotics previously known in sperm cell processing in examples 5 and 6 without itself damaging the sperm. Conventional antibiotics, such as Gentamicin, are limited in the dosages they can be applied to sperm in buffers or through other medias. Gentamicin, as an example, can become damaging to sperm membranes in dosages above conventional dosages. The bacterial infections found in Example 5 were surprising and were found even in the presence of such a conventional dosages of Gentamicin and Penicillin.

The addition of a quinolone, such as ciprofloxacin can be carried out at any variety of times throughout a sperm processing. For example, the sperm can be treated with quinolones at the time the sperm is obtained. Whether the sperm is obtained through thawing frozen straws or through the collection of ejaculate, the quinolone can be introduced by collecting directly into a buffer containing the quinolone or by mixing the sperm sample with a solution containing quinolones.

Once a sperm sample has been stained with a marker, such a fluorescent DNA selective dye, the sperm can be examined by flow cytometry. It should be appreciated that other methods for measuring and detecting molecular markers and/or fluorescent markers can be used; including, but not limited to, the use of a spectrophotometer and microfluidic chips and these should be considered embodiments of the disclosed methods. Flow cytometry can be used as described in U.S. Pat. No. 6,357,307, as referenced above, to determine the amount of DNA in each cell, and the cells can be separated based on this measurement.

Once the cells have been evaluated, they can be separated in a number of ways. U.S. Pat. No. 6,357,307 discusses the use of electromagnetic deflection used in conjunction with flow cytometry. However, embodiments of the current method also contemplate the use of microfluidic channels for separating the cells. U.S. Patent Application Publication 2006/0270021 and U.S. Pat. No. 7,298,478, the entire contents each are herby incorporated herein incorporated by reference, provide examples of microfluidic channels which could be used for the separation of sperm. Regardless of the method of separation, it may be desirable to reduce the DNA fragmentation in sorted sperm subpopulations in order to promote successful pregnancies and births. Embodiments presented herein relate to the modification to sperm handling processes in order to reduce the DNA fragmentation present after sex sorting or to reduce the rate at which DNA fragmentation increases after sex sorting.

In one aspect, the level of DNA fragmentation is improved with the use of quinolones which been found to be more effective than commonplace antibiotics such as gentamicin, without having a detrimental effect on sperm membranes that would accompany increased dosages of typical antibiotics. In another aspect, modifying the staining process has been shown to improve the DNA fragmentation in sex sorted sperm subpopulations by changing the pH at which certain staining steps are performed.

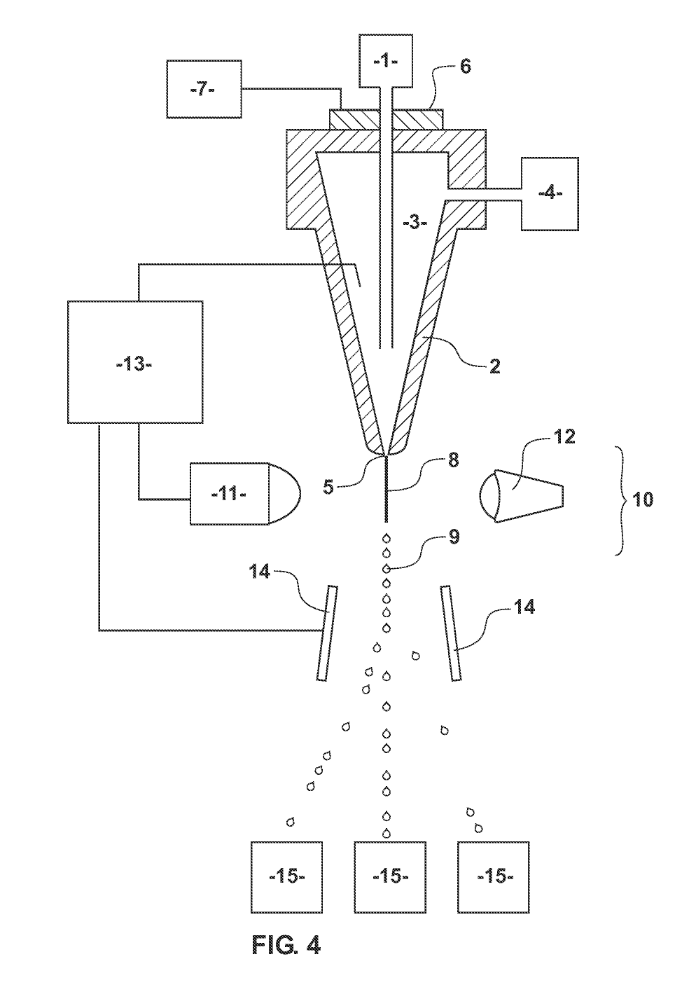

Turning now to FIG. 4, a system is illustrated for carrying out certain methods described herein. The system includes a cell source 1 for establishing a supply of cells for analysis and/or sorting. The cells are introduced into a nozzle 2 along with a sheath fluid 3 is introduced from a sheath fluid source 4. The sheath fluid 3 forms a sheath fluid environment around the cells as both are fed out of the nozzle 2 through a nozzle orifice 5.

The pressure with which fluids are supplied to the nozzle 2 affects the velocity of a stream 8 exiting the nozzle orifice 5. The stream 8 can further be controlled by an oscillator 6 through an oscillator controller 7 which produces pressure waves in nozzle 2 and the nozzle orifice 5. These pressure waves are transferred through the nozzle 4 and nozzle orifice 5 to the stream 8, resulting in the regular formation of droplets 9 at a break off point. The diameter of the nozzle orifice 5 and the frequency of the oscillator 6 can be coordinated to produce droplets which are large enough to entrain isolated cells.

The droplets 9 entraining individual cells can be analyzed and/or sorted based on the characteristics of the cells entrained in each of the droplets 9. As part of the flow cytometer, a cell sensing system 10 can be incorporated for making these distinctions. The cell sensing system 10 can include a detector or sensor 11 which responds to the cells contained within the stream 8. The cell sensing system 10 can cause an action depending on the relative presence or absence of a characteristic, such as a fertility characteristic. For example, the presence, absence or quantity of a DNA selective dye can be used in order to characterize cells as more having an X-chromosome or a Y-chromosome.

As one example, a DNA selective dye, such as a fluorochrome dye, can be bound to the DNA within the cell as a molecular marker. The DNA selective dye can be excited by an excitation device 12, such as a laser, which emits an irradiation beam causing the DNA selective dye to react or fluoresce. For the purpose of sex sorting sperm, each sperm can be stained with a DNA selective dye, such as Hoechst 33342. The total fluorescence of each passing cell dependents upon the amount of DNA contained within each cell, thereby providing a means for distinguishing X-chromosome bearing sperm from Y-chromosome bearing sperm. The sperm samples contain a number of dead or membrane compromised sperm which are not desirable for sorting. In order to remove the damaged sperm, a second staining step is employed which introduces quenching dye. Quenching dyes modify the interaction of the DNA selective dye and sperm with compromised membranes by dampening or reducing the detectable fluorescence from within the damaged sperm. In this way, damaged sperm does not produce a fluorescence which would be quantified as either X-chromosome bearing or Y-chromosome bearing. Instead, the low fluorescence emission characterizes sperm as damaged or dead.

The process for staining cells with the first fluorescent dye and the second quenching dye may contribute to the DNA fragmentation which has been shown to occur after sorting. In one embodiment presented herein, the second stain is applied in a second medium at an elevated pH, which can be a pH similar to the pH of a first medium containing the DNA selective dye. It should be appreciated that in the case of bovine the pH of the first and the second dye can be around about 7.4, but that staining conditions are known for staining different species sperm with the fluorescent dye, and that embodiments presented herein contemplate that those can be carried out in addition to the second dye at the same, or a similar, pH. Further aspects herein relate to coordinating the pH of the second medium with the first medium.

The fluorescence can be picked up by a sensor 11 and converted into an electrical signal. That electrical signal can be input into an analyzer 13 for making a determination based on the emitted fluorescence. The analyzer 13 can be coupled to a droplet charger for differentially charging the stream 8, and thus droplets 9 just prior to their break off. The timing of the detection and the charging is coordinated such that the stream is charged just prior to the break off of a droplet in containing the analyzed cell. Once the droplet is broken off, it retains the charge of the stream.

FIG. 4 also illustrates deflection plates 14 on either side of the nozzle 2 in order to direct cells into one of several possible trajectories. The deflection plates can be charged with opposite electrical fields, for example approximately +2500 Volts for the left hand plate and −2500 Volts right hand plate respectively. It should be appreciated that depending on the apparatus, the plates can be charged up to about 4000 Volts in either polarization. Those in the field familiar with the operation of flow cytometers can set up such deflection plates in any number of configurations using various charge configurations. For example, the faster the velocity of the flow stream the more voltage is required to pull a droplet onto a specific trajectory. As droplets fall between the deflection plates, they will be attracted towards the plate having an opposite charge, or fall straight downwards in the case where no charge is applied to a droplet 9. The collection containers 15 are illustrated as three containers for collecting droplets which: have not been charged, have been positively charged, and have been negatively charged. It should be appreciated that the arrangement of three containers 15 illustrated in FIG. 4 can be configured on the MoFlo SX, but that one of the illustrated streams is for waste, or empty droplets. Therefore, in order to sort a sample into three populations, an additional container is required for waste. The MoFlo SX XDP can be configured for an X-enriched stream, a Y-enriched stream, an unsorted live stream, and a waste stream including dead sperm.

Quenching dyes primarily serve to distinguish dead or membrane compromised sperm from otherwise healthy sperm in a sample. This is achieved by the accumulation of a quenching dye within the dead sperm. Flourochrome dyes, such as Hoechst 33342 will bind to the DNA of both dead and living sperm. However, only the dead or membrane compromised cells will have both the fluorochrome and the quenching dye within the membrane. When this dead or damaged sperm is excited with radiant energy, typically a laser operated at the UV wavelength, a portion of light emitted by the fluorochrome dye will be absorbed, or dampened, by the quenching dye within the membrane. In this way, dead cells are quenched so they only emit less intense signals in contrasted to the unquenched live sperm which produces more intense signals.

The quenching dye is specifically selected for its ability to absorb, or dampen, light emitted or fluoresced by the fluorochrome dye, which can lead to other problems in resolution. One problem arises with the use of traditional quenching dyes, such as red food dye 40, particularly in the use of flow cytometry, because in addition to the quenching dye associated with dead sperm, there is some quenching dye in the core stream. This loose or unassociated quenching dye then dampens the signals produced by fluorochromes in both living and dead cells. In short, this unassociated dye tends to dampen all the signals making the light of interest more difficult for the detectors to capture. This results in a loss of resolution at the detector. The difference between live cells and dead cells is generally still clearly distinguishable. However, this loss in resolution can affect the purity of sex sorting sperm since there is generally about a 2-4% difference in the amount of DNA in an X-chromosome bearing sperm and a Y-chromosome bearing sperm.

Surprisingly, it has been found that the yellow 6 food dye improves the resolution in flow cytometery for sperm sorting by providing an effective quenching for dead sperm and by disrupting less emitted light in the core stream. The yellow 6 food dye enters membrane compromised sperm in such a way that it largely dampens any fluorescence produced from fluorescent dyes on the same DNA. The yellow dye quenches the fluorochrome emitted from dead cells enough so that dead cells are clearly distinguished from live cells, but yellow dye in the core stream dampens less light meaning the resolution between X-chromosome bearing sperm and Y-chromosome bearing sperm is improved. In one embodiment, the amount of the fluorochrome dye can be reduced with the use of yellow 6 food dye as a quenching dye. It is well known that staining conditions for fluorochrome dyes are harsh to fragile sperm. Any steps which reduce the amount of fluorochrome dye required or the incubation time required for staining provides a substantial benefit to the long term health of the sperm. Therefore, yellow 6 food dye as a quencher could provide a substantial benefit by improving sorted sperm viability.

Similarly, there may be benefits to using any of a green food dye, an orange food dye, or a blue food dye as compared to these dyes in the prior art. Yellow food dye, as a quenching dye, could provide similar benefits in other instances presenting poor resolution. For example, thawed previously-frozen sperm can be more difficult to resolve than fresh and the use of a yellow dye as a quenching stain may improve the resolution. It should also be appreciated that red food dye could also be used at lower concentrations in order to achieve improved results as compared to the standard dosage of red food dye. However, increasing volume of red food dye has been shown to have a negative impact on sorting resolution.

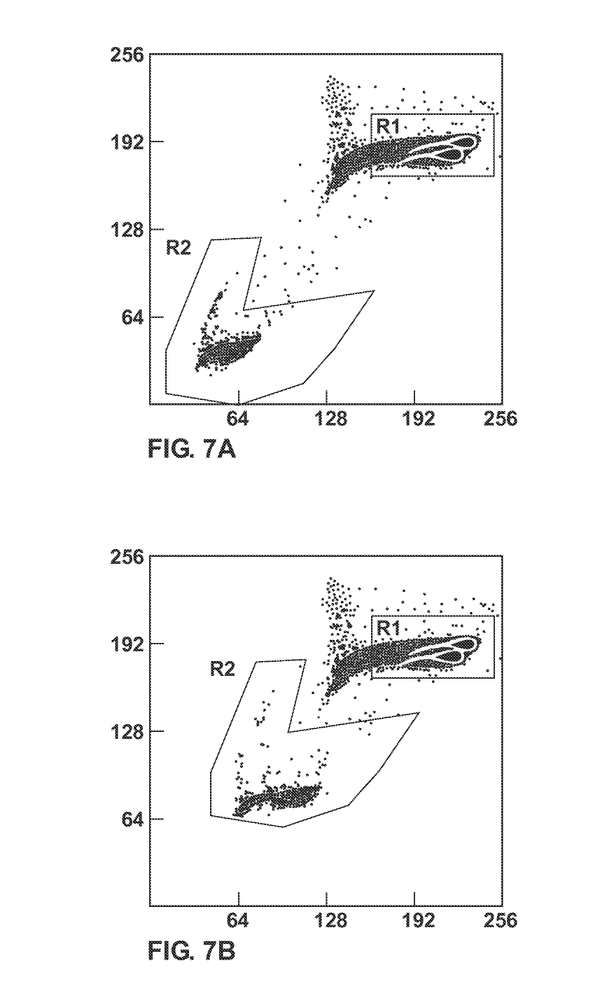

FIG. 7A illustrates a screenshot from a flow cytometer sex sorting sperm, and particularly illustrates the forward fluorescence of sperm in a running sample against the side fluorescence of sperm in a running sample. The sperm in this figure were stained with the fluorochrome dye, Hoechst 33342, and with red food dye 40. The region R2 in FIG. 7A represents sperm highly quenched with a red food dye. Because of the red food dye these quenched sperm do not emit an intense forward fluorescence or side fluorescence. In contrast, region R1 depicts the living sperm, unquenched, which are both properly orientated and alive to produce significant forward fluorescence and side fluorescence. These sperm in R1 are typically then gated for separation into an X-enriched and/or a Y-enriched sperm population. FIG. 7B illustrates a typical staining carried out on the same bull as FIG. 7A but with yellow dye 6 as a quencher. Contrasting FIG. 7A and FIG. 7B, it can be see that FIG. 7B still maintains a clear distinction between R1 and R2. The yellow 6 food dye further provides an advantage in the case of bulls presenting poor separation between live and dead sperm. Specifically, the yellow 6 food dye can be increased to concentrations of at least 400% while maintaining good resolution. In contrast, increasing red food dye over 100% very quickly impacts sorting resolution significantly.

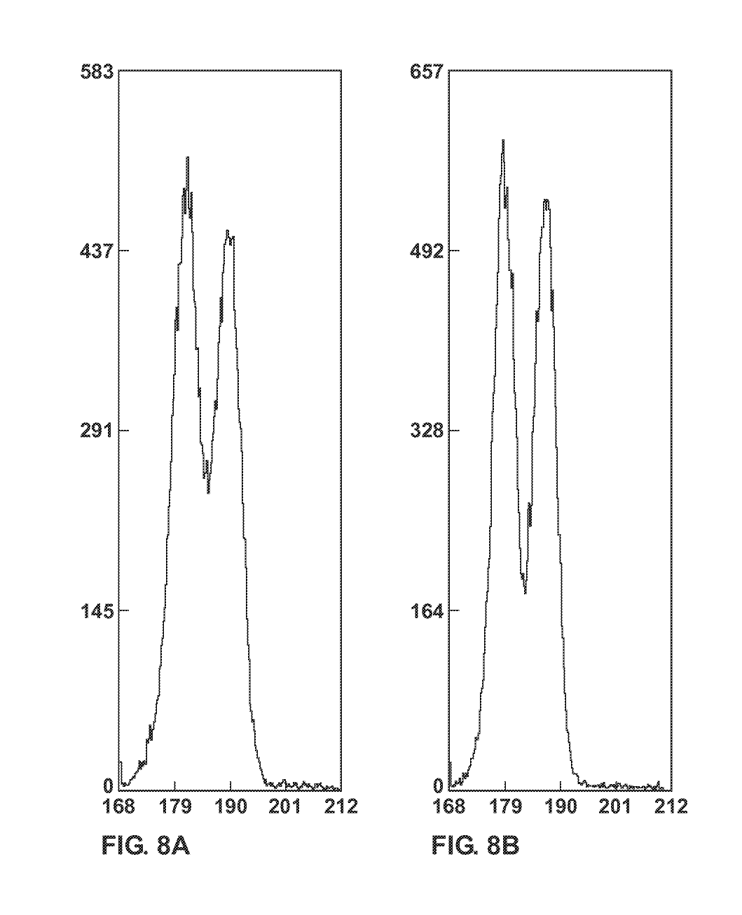

Now looking to FIG. 8A and 8B a single parameter histogram representing peak forward fluorescence for two sorts is illustrated. FIG. 8A corresponds to FIG. 7A which was processed with red food dye, and FIG. 8B corresponds to FIG. 7B which was processed with yellow food dye. In each, two distinct peaks can represent the population of sperm having each of X-chromosomes and Y-chromosomes. The more these peaks overlap, the lower the resolution is resulting in lower purities, and in most cases sorting speeds may have to be dropped in order to achieve acceptable purities with poor resolution.

While the invention has been described with respect to a limited number of embodiments, those skilled in the art, having benefit of this disclosure, will appreciate that other embodiments can be devised which do not depart from the scope of the invention as disclosed herein. Specifically, the treatment of a sperm sample with quinolones to reduce the levels of bacterial infection and DNA fragmentation in a given sample would be expected to work just as well in other types of reproductive cell samples such as suspensions of oocytes, embryos, and other related cell types. Numerous alternative embodiments could be implemented within the scope of the claims using current technology, or technology developed after the filing date of this patent.

Thus, many modifications and variations may be made to the process steps and structures described and illustrated in the enumerated embodiments. Accordingly, the scope of the invention should be limited only by the attached claims.

We claim:

1. A method for reducing DNA fragmentation in a sorted sperm sample comprising the steps of:
a. obtaining a sperm sample;
b. combining the sperm sample with a quinolone;
c. inhibiting bacterial growth in the sperm sample; and
d. sorting the sperm sample.

2. The method according to claim 1 wherein the quinolone comprises a fluoroquinolone.

3. The method according to claim 2 wherein the fluoroquinolone comprises ciprofloxacin.

4. The method according to claim 1 further comprising the step of: cryopreserving the sperm sample.

5. The method according to claim 1 further comprising the step of extending the sorted sperm sample with a buffer solution to form an extended sperm sample.

6. The method according to claim 5 wherein the step of combining the sperm sample with a quinolone further comprises: applying the quinolone to one selected from the group of: the sperm sample, the extended sperm sample, and the buffer solution.

7. (canceled)

8. (canceled)

9. The method according to claim 1 wherein the step of combining the sperm sample with the quinolone occurs substantially at the time the sperm is collected.

10. The method according to claim 4 further comprising the step of thawing the cryopreserved sperm sample.

11. The method according to claim 10 wherein the step of combining the sperm sample with a quinolone occurs after the step of thawing the cryopreserved sperm sample.

12. The method according to claim 1 wherein the quinolone is provided at a concentration between about 0.05 μg/ml and about 20 μg/ml, between about 0.2 μg/ml and about 5 μg/ml, and/or between about 0.1 μg/ml and about 2 μg/ml.

13. (canceled)

14. The method according to claim 1 wherein the step of sorting sperm further comprises the steps of:
a. differentiating sperm within the sperm sample based on a desired fertility characteristic; and
b. sorting sperm based on the desired fertility characteristic.

15. The method according to claim 14 wherein the step of sorting sperm based on the desired fertility characteristic further comprises: sex sorting sperm in the sperm sample for forming a gender enriched population of X-chromosome bearing sperm and/or a gender enriched population of Y-chromosome bearing sperm.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method for processing a sperm sample comprising the steps of:
a. obtaining a sperm sample;
b. staining the sperm sample with a DNA selective dye in a first medium having a pH;
c. coordinating the pH of a second medium including a second dye with the pH of the first medium; and
d. staining the sperm sample with the second dye in the second medium.

31. The method according to claim 30 further comprising the steps of:
a. sorting the stained sperm sample into distinct subpopulations according to the amount of DNA selective dye associated with each sperm; and
b. collecting at least one subpopulation of sperm based on the step of sorting.

32. The method according to claim 31 wherein the pH of the second medium is coordinated to be within 2 pH of the first medium and/or 1 pH of the first medium.

33. The method of claim 32 wherein the pH of the second medium is coordinated to be about the same as the pH of the first medium.

34. The method of claim 31 wherein the second dye is a quenching dye.

35. The method of claim 34 wherein the second medium comprises a red TALP.

36. The method of claim 35 wherein the red TALP comprises a TALP based buffer and red food dye.

37. The method of claim 34 wherein the quenching dye is one selected from the group consisting of: red food dye, yellow food dye, percoll, propidum iodide, and trypan blue.

38. The method of claim 30 wherein the step of coordinating the pH of the second medium further comprises the step of adjusting the pH of the second medium to between about 5.5 and about 7.4, between about 6.4 and about 7.4, and/or between about 5.5 and about 6.4.

39. (canceled)

40. (canceled)

41. The method according to claim 34 wherein the sperm sample comprises bovine sperm and the pH of both the first dye and the second dye are about 7.4.

42. The method according to claim 34 wherein the pH of the second medium is greater than 5.5.

43. The method according to claim 34 wherein the sperm is sorted on the basis of carrying an X-chromosome or a Y-chromosome.

44. The method according to claim 34 wherein the first stain comprises a fluorescent DNA selective dye.

45. (canceled)

46. The method of claim 34 wherein the step of coordinating the pH of the second medium further comprises the step of adjusting the pH of the second medium based upon the pH of the first medium and the pH of the sperm sample.

47. The method of claim 46 wherein the step of coordinating the pH of the second medium further comprises the step of adjusting the pH of the second medium to achieve a sperm sample pH between 6.6 and 7.2.

48. (canceled)

49. The method according to claim 34 wherein the step of staining with the first medium and the second medium is performed at the same time as a single pH adjusting event.

50. (canceled)

51. (canceled)

52. (canceled)

53. (canceled)

54. (canceled)

55. (canceled)

56. (canceled)

57. (canceled)

58. (canceled)

59. (canceled)

60. A method of staining sperm comprising the steps of:
a. obtaining sperm;
b. incubating the sperm under controlled staining conditions with a fluorochrome dye;
c. adding a quenching dye to the sperm, wherein the quenching dye is selected from the group comprising: yellow food dye, orange food dye, green food dye and combinations thereof.

61. The method according to claim 60 wherein the quenching dye comprises yellow food dye.

62. The method according to claim 61 wherein the yellow food dye comprises yellow food dye Number 6.

63. The method according to claim 61 wherein yellow food dye provides higher sorting resolutions than conventional red food dye quenchers.

64. The method of claim 60 wherein the controlled staining conditions comprise:
a. incubating at a temperature between 30 and 39° C.;
b. incubating at a pH between 7.0 and 7.4; and
c. incubating at a time between 20 minutes and an hour.

65. (canceled)

66. The method according to claim 60 wherein the stained sperm is sorted in a microfluidic device.

67. (canceled)

68. The method according to claim 60 wherein the step of adding the quenching dye occurs after the step of incubating at controlled staining conditions.

69. The method of claim 60 wherein the step of adding the quenching dye occurs during the incubation period with the fluorochrome dye.

70. A method of sorting sperm stained by the process of claim 60 further comprising the steps of:
a. flowing the stained sperm through a microfluidic device;
b. exposing the stained sperm to radiant energy to excite the fluouchrome dye;
c. distinguishing between sperm having an X-chromosome and Y-chromosome based upon the energy fluoresced from the stained sperm in response to the radiant energy.
